(19) United States
(12) Patent Application Publication
(10) Pub. No.: US 2015/0176033 A1
(43) Pub. Date: Jun. 25, 2015

(54) REACTIVE OXYGEN SPECIES-RESISTANT MICROORGANISMS

(71) Applicant: JOULE UNLIMITED TECHNOLOGIES, INC., Bedford, MA (US)

(72) Inventors: Michael Connor, Somerville, MA (US); Frank A. Skraly, Watertown, MA (US)

(73) Assignee: Joule Unlimited Technologies ,Inc. a corporation

(21) Appl. No.: 14/367,866

(22) PCT Filed: Dec. 21, 2012
(86) PCT No.: PCT/US2012/071250

§ 371 Date: Jun. 20, 2014

Related U.S. Application Data
(60) Provisional application No. 61/580,094, filed on Dec. 23, 2011.

(51) Int. Cl. C12P 7/06; C12N 15/74; C12N 9/08

(52) U.S. Cl. CPC C12P 7/065; C12N 9/0065; C12N 15/74; C12Y 111/01006; C12Y 111/01007; C12Y 111/01021

(57) ABSTRACT

The present disclosure identifies pathways and mechanisms to confer resistance to reactive oxygen species, e.g., hydrogen peroxide, to photoautotrophic organisms. The use of such organisms in host cell culture systems with high levels of reactive oxygen species, such as host cell culture systems decontaminated with hydrogen peroxide, is contemplated.

### CROSS-REFERENCE TO RELATED APPLICATIONS

This application claims the benefit of U.S. Provisional Patent Application No. 61/580,094, filed Dec. 23, 2011, the disclosure of which is incorporated herein by reference.

### SEQUENCE LISTING

The instant application contains a Sequence Listing which has been submitted in ASCII format via EFS-Web and is hereby incorporated by reference in its entirety. Said ASCII copy, created on Dec. 20, 2012, is named 21978PCT_CRF_SequenceListing.txt and is 14,384 bytes in size.

### BACKGROUND

Recombinant photosynthetic microorganisms have been engineered to produce carbon-based products of interest. To maximize yields of the recombinant photosynthetic microorganisms, their productivity and viability must be optimized. Contamination control of the microorganism culture is also crucial, as contaminants may compete directly for nutrients or consume the desired end product. Additionally, contamination can inhibit the production of hydrocarbons by recombinant photosynthetic microorganisms. What is needed therefore is a system to inhibit contamination, and a recombinant photosynthetic microorganism engineered to maximize viability in this system.

### SUMMARY

Disclosed herein are compositions and methods to produce a carbon-based product of interest. In one embodiment, a method to produce a carbon-based product of interest is provided, comprising culturing an engineered cyanobacterial cell in a cell culture in the presence of CO₂ and light under conditions suitable to produce a carbon-based product of interest, wherein the engineered cyanobacterial cell comprises a recombinant nucleic acid encoding an enzyme classified under an Enzyme Commission number selected from EC 1.11.1.6, EC 1.11.1.7, and EC 1.11.1.21.

In another embodiment, the enzyme encoded by the recombinant nucleic acid has catalase activity. In another embodiment, the enzyme has peroxidase activity. In some embodiments, the enzyme is selected from KatG and KatE. In another embodiment, the enzyme comprises an amino acid sequence at least 90% identical to SEQ ID NO: 3. In other embodiments, the enzyme comprises an amino acid sequence at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to SEQ ID NO: 3. In some embodiments, the enzyme comprises an amino acid sequence at least 90% identical to SEQ ID NO: 4. In other embodiments, the enzyme comprises an amino acid sequence at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to SEQ ID NO: 4.

In some embodiments, the cell culture further comprises hydrogen peroxide. In certain embodiments, the hydrogen peroxidie in the cell culture is at a concentration of from 0.1 to 50 mM. In other embodiments, the cell culture comprises a reagent to mitigate contamination of the cell culture. In some embodiments, the cell culture is exposed to diurnal light conditions.

In an embodiment, the engineered cyanobacterial cell has the same or a higher rate of ethanol production in the cell culture than an otherwise identical cyanobacterial cell lacking the recombinant nucleic acid. In an embodiment, the production rate of ethanol of a culture of the engineered cyanobacterial cells is greater than a rate selected from: 50, 100, 150, 200, 250, 300, 350, and 400 mg*L⁻¹*day⁻¹. In some embodiments, the production rate of ethanol of a culture of the engineered cyanobacterial cells ranges from 50-100 mg*L⁻¹*day⁻¹, 100-150 mg*L⁻¹*day⁻¹, 150-200 mg*L⁻¹*day⁻¹, 250-300 mg*L⁻¹*day⁻¹, 300-350 mg*L⁻¹*day⁻¹, or 350-400 mg*L⁻¹*day⁻¹. In an embodiment, the production rate of ethanol of a culture of the engineered byanobacterial cells is greater than a rate selected from: 13, 14, 15, and 16 mg*L⁻¹*h⁻¹. In some embodiments, the production rate of ethanol of a culture of the engineered byanobacterial cells is between: 13-14 mg*L⁻¹*h⁻¹, 14-15 mg*L⁻¹*h⁻¹, 15-16 mg*L⁻¹*h⁻¹, and 16-17 mg*L⁻¹*h⁻¹. In some embodiments, the mass of ethanol produced by a culture of the engineered cyanobacterial cells per volume of the culture is greater than an amount selected from: 1.5, 2, 2.5, 3, 3.5, 4, 4.5, 5, 5.5, 6, 6.5, 7, 7.5, and 8 g/L. In some embodiments, the mass of ethanol produced by a culture of the engineered cyanobacterial cells per volume of the culture ranges from 1.5-2 g/L, 2-2.5 g/L, 2.5-3 g/L, 3-3.5 g/L, 3.5-4 g/L, 4-4.5 g/L, 4.5-5 g/L, 5-5.5 g/L, 5.5-6 g/L, 6-6.5 g/L, 6.5-7 g/L, 7-7.5 g/L, 7.5-8 g/L, or 8-8.5 g/L. In some embodiments, this amount of ethanol is produced during up to 30 days of culture. In some embodiments, this amount of ethanol is produced over the course of 15-20 days, 20-25 days, 25-30 days, or 30-35 days.

In one embodiment, the cyanobacterial cell is an ethanologen. In some embodiments, the cyanobacterial cell is a Synechococcus species. In certain embodiments, the Synechococcus species is Synechococcus PCC 7002.

Also provided herein is a method for conferring hydrogen peroxide resistance to a parent cyanobacterial cell, the method comprising transforming the parent cyanobacterial cell with a nucleic acid encoding an enzyme selected from KatG and KatE, resulting in an engineered cyanobacterial cell, and culturing the engineered cyanobacterial cell in a medium comprising hydrogen peroxide.

In an embodiment, the hydrogen peroxide in said medium is at a concentration of 0.1 to 50 mM. In an embodiment, the parent cyanobacterial cell is an ethanologen. In some embodiments, the parent cyanobacterial cell is a Synechococcus species. In certain embodiments, the Synechococcus species is Synechococcus PCC 7002.

In an embodiment, the enzyme comprises an amino acid sequence at least 90% identical to SEQ ID NO: 3. In another embodiment, the enzyme comprises an amino acid sequence at least 90% identical to SEQ ID NO: 4. In some embodiments, the engineered cyanobacterial cell has an increased or identical rate of ethanol production as compared to the parent cyanobacterial cell.

Also disclosed herein is an engineered host cell comprising a recombinant gene encoding a first enzyme classified under an Enzyme Commission number selected from EC 1.11.1.6, EC 1.11.1.7, and EC 1.11.1.21, wherein said host cell further comprises a recombinant pyruvate decarboxylase or a recombinant alcohol dehydrogenase.

In some embodiments, host cell is a cyanobacterium. In certain embodiments, the cyanobacterium is Synechococcus PCC 7002. In some embodiments, the first enzyme is selected from KatG and KatE. In an embodiment, the first enzyme comprises an amino acid sequence at least 90% identical to SEQ ID NO: 3. In another embodiment, the first enzyme comprises an amino acid sequence at least 90% identical to SEQ ID NO: 4.

These and other embodiments of the invention are further described in the Figures, Description, Examples and Claims, herein.

### BRIEF DESCRIPTION OF THE FIGURES

FIG. 1 shows growth (A) and ethanol production (B) in cultures of JCC1510 (parent) and JCC2979 (engineered to express heterologous katG) strains of ethanologenic cyanobacteria exposed to continuous light.

FIG. 2 shows growth (A) and ethanol production (B) in cultures of JCC1510 (parent) and JCC2979 (engineered to express heterologous katG) strains of ethanologenic cyanobacteria exposed to natural light conditions (12 hour light/dark cycles).

FIG. 3 shows growth in cultures of JCC1581 (parent) and JCC3351 (engineered to express heterologous katG) strains of ethanologenic cyanobacteria exposed to selected levels of hydrogen peroxide (H₂O₂). Panels A and B show the result of culture growth from a starting point of OD₇₃₀=0.1. Panels C and D show the result of culture growth from a starting point of OD₇₃₀=3.0. Panels B and D show normalized OD730 values calculated by dividing each measured OD₇₃₀ value after 24 hours with the highest absolute OD₇₃₀ value after 24 hours.

FIG. 4 shows growth (A) and ethanol production (B) in cultures of JCC1581 (parent) and JCC3351 (engineered to express heterologous katG) strains of ethanologenic cyanobacteria exposed to natural light conditions (12 hour light/dark cycles).

### DETAILED DESCRIPTION

Unless otherwise defined herein, scientific and technical terms shall have the meanings that are commonly understood by those of ordinary skill in the art. Further, unless otherwise required by context, singular terms shall include the plural and plural terms shall include the singular. Generally, nomenclatures used in connection with, and techniques of, biochemistry, enzymology, molecular and cellular biology, microbiology, genetics and protein and nucleic acid chemistry and hybridization described herein are those well known and commonly used in the art.

The methods and techniques of the present disclosure are generally performed according to conventional methods well known in the art and as described in various general and more specific references that are cited and discussed throughout the present specification unless otherwise indicated. See, e.g., Sambrook et al., Molecular Cloning: A Laboratory Manual, 2d ed., Cold Spring Harbor Laboratory Press, Cold Spring Harbor, N.Y. (1989); Ausubel et al., Current Protocols in Molecular Biology, Greene Publishing Associates (1992, and Supplements to 2002); Harlow and Lane, Antibodies: A Laboratory Manual, Cold Spring Harbor Laboratory Press, Cold Spring Harbor, N.Y. (1990); Taylor and Drickamer, Introduction to Glycobiology, Oxford Univ. Press (2003); Worthington Enzyme Manual, Worthington Biochemical Corp., Freehold, N.J.; Handbook of Biochemistry: Section A Proteins, Vol I, CRC Press (1976); Handbook of Biochemistry: Section A Proteins, Vol II, CRC Press (1976); Essentials of Glycobiology, Cold Spring Harbor Laboratory Press (1999).

One skilled in the art will also recognize, in light of the teachings herein, that the methods and compositions described herein for use in particular organisms, e.g., cyanobacteria, are also applicable other organisms, e.g., gram-negative bacteria such as E. coli. For example, a chimeric integral plasma membrane protein for facilitating alkane efflux in E. coli could be designed by fusing a pseudo leader sequence derived from E. coli or a related bacterium to a heterologous integral plasma membrane protein.

The following terms, unless otherwise indicated, shall be understood to have the following meanings:

The term “polynucleotide” or “nucleic acid molecule” refers to a polymeric form of nucleotides of at least 10 bases in length. The term includes DNA molecules (e.g., cDNA or genomic or synthetic DNA) and RNA molecules (e.g., mRNA or synthetic RNA), as well as analogs of DNA or RNA containing non-natural nucleotide analogs, non-native internucleoside bonds, or both. The nucleic acid can be in any topological conformation. For instance, the nucleic acid can be single-stranded, double-stranded, triple-stranded, quadruplexed, partially double-stranded, branched, hairpinned, circular, or in a padlocked conformation.

Unless otherwise indicated, and as an example for all sequences described herein under the general format “SEQ ID NO:”, “nucleic acid comprising SEQ ID NO:1” refers to a nucleic acid, at least a portion of which has either (i) the sequence of SEQ ID NO:1, or (ii) a sequence complementary to SEQ ID NO:1. The choice between the two is dictated by the context. For instance, if the nucleic acid is used as a probe, the choice between the two is dictated by the requirement that the probe be complementary to the desired target.

An “isolated” RNA, DNA or a mixed polymer is one which is substantially separated from other cellular components that naturally accompany the native polynucleotide in its natural host cell, e.g., ribosomes, polymerases and genomic sequences with which it is naturally associated.

As used herein, an “isolated” organic molecule (e.g., an alkane, alkene, or alkanal) is one which is substantially separated from the cellular components (membrane lipids, chromosomes, proteins) of the host cell from which it originated, or from the medium in which the host cell was cultured. The term does not require that the biomolecule has been separated from all other chemicals, although certain isolated biomolecules may be purified to near homogeneity.

The term “recombinant” refers to a biomolecule, e.g., a gene or protein, that (1) has been removed from its naturally occurring environment, (2) is not associated with all or a portion of a polynucleotide in which the gene is found in nature, (3) is operatively linked to a polynucleotide which it is not linked to in nature, or (4) does not occur in nature. The term “recombinant” can be used in reference to cloned DNA isolates, chemically synthesized polynucleotide analogs, or polynucleotide analogs that are biologically synthesized by heterologous systems, as well as proteins and/or mRNAs encoded by such nucleic acids.

As used herein, an endogenous nucleic acid sequence in the genome of an organism (or the encoded protein product of that sequence) is deemed “recombinant” herein if a heterologous sequence is placed adjacent to the endogenous nucleic acid sequence, such that the expression of this endogenous nucleic acid sequence is altered. In this context, a heterologous sequence is a sequence that is not naturally adjacent to the endogenous nucleic acid sequence, whether or not the heterologous sequence is itself endogenous (originating from the same host cell or progeny thereof) or exogenous (originating from a different host cell or progeny thereof). By way of example, a promoter sequence can be substituted (e.g., by homologous recombination) for the native promoter of a gene in the genome of a host cell, such that this gene has an altered expression pattern. This gene would now become “recombinant” because it is separated from at least some of the sequences that naturally flank it.

A nucleic acid is also considered “recombinant” if it contains any modifications that do not naturally occur to the corresponding nucleic acid in a genome. For instance, an endogenous coding sequence is considered “recombinant” if it contains an insertion, deletion or a point mutation introduced artificially, e.g., by human intervention. A “recombinant nucleic acid” also includes a nucleic acid integrated into a host cell chromosome at a heterologous site and a nucleic acid construct present as an episome.

As used herein, the phrase “degenerate variant” of a reference nucleic acid sequence encompasses nucleic acid sequences that can be translated, according to the standard genetic code, to provide an amino acid sequence identical to that translated from the reference nucleic acid sequence. The term “degenerate oligonucleotide” or “degenerate primer” is used to signify an oligonucleotide capable of hybridizing with target nucleic acid sequences that are not necessarily identical in sequence but that are homologous to one another within one or more particular segments.

The term “percent sequence identity” or “identical” in the context of nucleic acid sequences refers to the residues in the two sequences which are the same when aligned for maximum correspondence. The length of sequence identity comparison may be over a stretch of at least about nine nucleotides, usually at least about 20 nucleotides, more usually at least about 24 nucleotides, typically at least about 28 nucleotides, more typically at least about 32 nucleotides, and in some instances at least about 36 or more nucleotides. There are a number of different algorithms known in the art which can be used to measure nucleotide sequence identity. For instance, polynucleotide sequences can be compared using FASTA, Gap or Bestfit, which are programs in Wisconsin Package Version 10.0, Genetics Computer Group (GCG), Madison, Wis. FASTA provides alignments and percent sequence identity of the regions of the best overlap between the query and search sequences. Pearson, Methods Enzymol. 183:63-98 (1990) (hereby incorporated by reference in its entirety). For instance, percent sequence identity between nucleic acid sequences can be determined using FASTA with its default parameters (a word size of 6 and the NOPAM factor for the scoring matrix) or using Gap with its default parameters as provided in GCG Version 6.1, herein incorporated by reference. Alternatively, sequences can be compared using the computer program, BLAST (Altschul et al., J. Mol. Biol. 215:403-410 (1990); Gish and States, Nature Genet. 3:266-272 (1993); Madden et al., Meth. Enzymol. 266:131-141 (1996); Altschul et al., Nucleic Acids Res. 25:3389-3402 (1997); Zhang and Madden, Genome Res. 7:649-656 (1997)), especially blastp or tblastn (Altschul et al., Nucleic Acids Res. 25:3389-3402 (1997)).

The term “substantial homology” or “substantial similarity,” when referring to a nucleic acid or fragment thereof, indicates that, when optimally aligned with appropriate nucleotide insertions or deletions with another nucleic acid (or its complementary strand), there is nucleotide sequence identity in at least about 76%, 80%, 85%, preferably at least about 90%, and more preferably at least about 95%, 96%, 97%, 98% or 99% of the nucleotide bases, as measured by any well-known algorithm of sequence identity, such as FASTA, BLAST or Gap, as discussed above.

Alternatively, substantial homology or similarity exists when a nucleic acid or fragment thereof hybridizes to another nucleic acid, to a strand of another nucleic acid, or to the complementary strand thereof, under stringent hybridization conditions. “Stringent hybridization conditions” and “stringent wash conditions” in the context of nucleic acid hybridization experiments depend upon a number of different physical parameters. Nucleic acid hybridization will be affected by such conditions as salt concentration, temperature, solvents, the base composition of the hybridizing species, length of the complementary regions, and the number of nucleotide base mismatches between the hybridizing nucleic acids, as will be readily appreciated by those skilled in the art. One having ordinary skill in the art knows how to vary these parameters to achieve a particular stringency of hybridization.

In general, “stringent hybridization” is performed at about 25° C. below the thermal melting point (Tₘ) for the specific DNA hybrid under a particular set of conditions. “Stringent washing” is performed at temperatures about 5° C. lower than the Tₘ for the specific DNA hybrid under a particular set of conditions. The Tₘ is the temperature at which 50% of the target sequence hybridizes to a perfectly matched probe. See Sambrook et al., Molecular Cloning: A Laboratory Manual, 2d ed., Cold Spring Harbor Laboratory Press, Cold Spring Harbor, N.Y. (1989), page 9.51, hereby incorporated by reference. For purposes herein, “stringent conditions” are defined for solution phase hybridization as aqueous hybridization (i.e., free of formamide) in 6×SSC (where 20×SSC contains 3.0 M NaCl and 0.3 M sodium citrate), 1% SDS at 65° C. for 8-12 hours, followed by two washes in 0.2×SSC, 0.1% SDS at 65° C. for 20 minutes. It will be appreciated by the skilled worker that hybridization at 65° C. will occur at different rates depending on a number of factors including the length and percent identity of the sequences which are hybridizing.

The nucleic acids (also referred to as polynucleotides) of this present disclosure may include both sense and antisense strands of RNA, cDNA, genomic DNA, and synthetic forms and mixed polymers of the above. They may be modified chemically or biochemically or may contain non-natural or derivatized nucleotide bases, as will be readily appreciated by those of skill in the art. Such modifications include, for example, labels, methylation, substitution of one or more of the naturally occurring nucleotides with an analog, internucleotide modifications such as uncharged linkages (e.g., methyl phosphonates, phosphotriesters, phosphoramidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.) Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule. Other modifications can include, for example, analogs in which the ribose ring contains a bridging moiety or other structure such as the modifications found in “locked” nucleic acids.

The term “mutated” when applied to nucleic acid sequences means that nucleotides in a nucleic acid sequence may be inserted, deleted or changed compared to a reference nucleic acid sequence. A single alteration may be made at a locus (a point mutation) or multiple nucleotides may be inserted, deleted or changed at a single locus. In addition, one or more alterations may be made at any number of loci within a nucleic acid sequence. A nucleic acid sequence may be mutated by any method known in the art including but not limited to mutagenesis techniques such as “error-prone PCR” (a process for performing PCR under conditions where the copying fidelity of the DNA polymerase is low, such that a high rate of point mutations is obtained along the entire length of the PCR product; see, e.g., Leung et al., Technique, 1:11-15 (1989) and Caldwell and Joyce, PCR Methods Applic. 2:28-33 (1992)); and “oligonucleotide-directed mutagenesis” (a process which enables the generation of site-specific mutations in any cloned DNA segment of interest; see, e.g., Reidhaar-Olson and Sauer, Science 241:53-57 (1988)).

The term “attenuate” as used herein generally refers to a functional deletion, including a mutation, partial or complete deletion, insertion, or other variation made to a gene sequence or a sequence controlling the transcription of a gene sequence, which reduces or inhibits production of the gene product, or renders the gene product non-functional. In some instances a functional deletion is described as a knockout mutation. Attenuation also includes amino acid sequence changes by altering the nucleic acid sequence, placing the gene under the control of a less active promoter, down-regulation, expressing interfering RNA, ribozymes or antisense sequences that target the gene of interest, or through any other technique known in the art. In one example, the sensitivity of a particular enzyme to feedback inhibition or inhibition caused by a composition that is not a product or a reactant (non-pathway specific feedback) is lessened such that the enzyme activity is not impacted by the presence of a compound. In other instances, an enzyme that has been altered to be less active can be referred to as attenuated.

The term “deletion” refers to the removal of one or more nucleotides from a nucleic acid molecule or one or more amino acids from a protein, the regions on either side being joined together.

The term “knock out” refers to a gene whose level of expression or activity has been reduced to zero. In some examples, a gene is knocked-out via deletion of some or all of its coding sequence. In other examples, a gene is knocked-out via introduction of one or more nucleotides into its open reading frame, which results in translation of a non-sense or otherwise non-functional protein product.

The term “vector” as used herein is intended to refer to a nucleic acid molecule capable of transporting another nucleic acid to which it has been linked. One type of vector is a “plasmid,” which generally refers to a circular double stranded DNA loop into which additional DNA segments may be ligated, but also includes linear double-stranded molecules such as those resulting from amplification by the polymerase chain reaction (PCR) or from treatment of a circular plasmid with a restriction enzyme. Other vectors include cosmids, bacterial artificial chromosomes (BAC) and yeast artificial chromosomes (YAC). Another type of vector is a viral vector, wherein additional DNA segments may be ligated into the viral genome (discussed in more detail below). Certain vectors are capable of autonomous replication in a host cell into which they are introduced (e.g., vectors having an origin of replication which functions in the host cell). Other vectors can be integrated into the genome of a host cell upon introduction into the host cell, and are thereby replicated along with the host genome. Moreover, certain vectors are capable of directing the expression of genes to which they are operatively linked. Such vectors are referred to herein as “recombinant expression vectors” (or simply “expression vectors”).

“Operatively linked” or “operably linked” expression control sequences refers to a linkage in which the expression control sequence is contiguous with the gene of interest to control the gene of interest, as well as expression control sequences that act in trans or at a distance to control the gene of interest.

The term “expression control sequence” as used herein refers to polynucleotide sequences which are necessary to affect the expression of coding sequences to which they are operatively linked. Expression control sequences are sequences which control the transcription, post-transcriptional events and translation of nucleic acid sequences. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (e.g., ribosome binding sites); sequences that enhance protein stability; and when desired, sequences that enhance protein secretion. The nature of such control sequences differs depending upon the host organism; in prokaryotes, such control sequences generally include promoter, ribosomal binding site, and transcription termination sequence. The term “control sequences” is intended to include, at a minimum, all components whose presence is essential for expression, and can also include additional components whose presence is advantageous, for example, leader sequences and fusion partner sequences.

The term “recombinant host cell” (or simply “host cell”), as used herein, is intended to refer to a cell into which a recombinant vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. A recombinant host cell may be an isolated cell or cell line grown in culture or may be a cell which resides in a living tissue or organism.

The term “peptide” as used herein refers to a short polypeptide, e.g., one that is typically less than about 50 amino acids long and more typically less than about 30 amino acids long. The term as used herein encompasses analogs and mimetics that mimic structural and thus biological function.

The term “polypeptide” encompasses both naturally-occurring and non-naturally-occurring proteins, and fragments, mutants, derivatives and analogs thereof. A polypeptide may be monomeric or polymeric. Further, a polypeptide may comprise a number of different domains each of which has one or more distinct activities.

The term “isolated protein” or “isolated polypeptide” is a protein or polypeptide that by virtue of its origin or source of derivation (1) is not associated with naturally associated components that accompany it in its native state, (2) exists in a purity not found in nature, where purity can be adjudged with respect to the presence of other cellular material (e.g., is free of other proteins from the same species) (3) is expressed by a cell from a different species, or (4) does not occur in nature (e.g., it is a fragment of a polypeptide found in nature or it includes amino acid analogs or derivatives not found in nature or linkages other than standard peptide bonds). Thus, a polypeptide that is chemically synthesized or synthesized in a cellular system different from the cell from which it naturally originates will be “isolated” from its naturally associated components. A polypeptide or protein may also be rendered substantially free of naturally associated components by isolation, using protein purification techniques well known in the art. As thus defined, “isolated” does not necessarily require that the protein, polypeptide, peptide or oligopeptide so described has been physically removed from its native environment.

The term “polypeptide fragment” as used herein refers to a polypeptide that has a deletion, e.g., an amino-terminal and/or carboxy-terminal deletion compared to a full-length polypeptide. In one aspect, the polypeptide fragment is a contiguous sequence in which the amino acid sequence of the fragment is identical to the corresponding positions in the naturally-occurring sequence. Fragments typically are at least 5, 6, 7, 8, 9 or 10 amino acids long, preferably at least 12, 14, 16 or 18 amino acids long, more preferably at least 20 amino acids long, more preferably at least 25, 30, 35, 40 or 45, amino acids, even more preferably at least 50 or 60 amino acids long, and even more preferably at least 70 amino acids long.

A “modified derivative” refers to polypeptides or fragments thereof that are substantially homologous in primary structural sequence but which include, e.g., in vivo or in vitro chemical and biochemical modifications or which incorporate amino acids that are not found in the native polypeptide. Such modifications include, for example, acetylation, carboxylation, phosphorylation, glycosylation, ubiquitination, labeling, e.g., with radionuclides, and various enzymatic modifications, as will be readily appreciated by those skilled in the art. A variety of methods for labeling polypeptides and of substituents or labels useful for such purposes are well known in the art, and include radioactive isotopes such as ¹²⁵I, ³²P, ³⁵S, and ³H, ligands which bind to labeled antiligands (e.g., antibodies), fluorophores, chemiluminescent agents, enzymes, and antiligands which can serve as specific binding pair members for a labeled ligand. The choice of label depends on the sensitivity required, ease of conjugation with the primer, stability requirements, and available instrumentation. Methods for labeling polypeptides are well known in the art. See, e.g., Ausubel et al., Current Protocols in Molecular Biology, Greene Publishing Associates (1992, and Supplements to 2002) (hereby incorporated by reference).

The term “fusion protein” refers to a polypeptide comprising a polypeptide or fragment coupled to heterologous amino acid sequences. Fusion proteins are useful because they can be constructed to contain two or more desired functional elements from two or more different proteins. A fusion protein comprises at least 10 contiguous amino acids from a polypeptide of interest, more preferably at least 20 or 30 amino acids, even more preferably at least 40, 50 or 60 amino acids, yet more preferably at least 75, 100 or 125 amino acids. Fusions that include the entirety of the proteins of the present disclosure have particular utility. The heterologous polypeptide included within the fusion protein of the present disclosure is at least 6 amino acids in length, often at least 8 amino acids in length, and usefully at least 15, 20, and 25 amino acids in length. Fusions that include larger polypeptides, such as an IgG Fc region, and even entire proteins, such as the green fluorescent protein (“GFP”) chromophore-containing proteins, have particular utility. Fusion proteins can be produced recombinantly by constructing a nucleic acid sequence which encodes the polypeptide or a fragment thereof in frame with a nucleic acid sequence encoding a different protein or peptide and then expressing the fusion protein. Alternatively, a fusion protein can be produced chemically by crosslinking the polypeptide or a fragment thereof to another protein.

As used herein, the term “antibody” refers to a polypeptide, at least a portion of which is encoded by at least one immunoglobulin gene, or fragment thereof, and that can bind specifically to a desired target molecule. The term includes naturally-occurring forms, as well as fragments and derivatives.

Fragments within the scope of the term “antibody” include those produced by digestion with various proteases, those produced by chemical cleavage and/or chemical dissociation and those produced recombinantly, so long as the fragment remains capable of specific binding to a target molecule. Among such fragments are Fab, Fab′, Fv, F(ab′)₂, and single chain Fv (scFv) fragments.

Derivatives within the scope of the term include antibodies (or fragments thereof) that have been modified in sequence, but remain capable of specific binding to a target molecule, including: interspecies chimeric and humanized antibodies; antibody fusions; heteromeric antibody complexes and antibody fusions, such as diabodies (bispecific antibodies), single-chain diabodies, and intrabodies (see, e.g., Intracellular Antibodies: Research and Disease Applications, (Marasco, ed., Springer-Verlag New York, Inc., 1998), the disclosure of which is incorporated herein by reference in its entirety).

As used herein, antibodies can be produced by any known technique, including harvest from cell culture of native B lymphocytes, harvest from culture of hybridomas, recombinant expression systems and phage display.

The term “non-peptide analog” refers to a compound with properties that are analogous to those of a reference polypeptide. A non-peptide compound may also be termed a “peptide mimetic” or a “peptidomimetic.” See, e.g., Jones, Amino Acid and Peptide Synthesis, Oxford University Press (1992); Jung, Combinatorial Peptide and Nonpeptide Libraries: A Handbook, John Wiley (1997); Bodanszky et al., Peptide Chemistry—A Practical Textbook, Springer Verlag (1993); Synthetic Peptides: A Users Guide, (Grant, ed., W. H. Freeman and Co., 1992); Evans et al., J. Med. Chem. 30:1229 (1987); Fauchere, J. Adv. Drug Res. 15:29 (1986); Veber and Freidinger, Trends Neurosci., 8:392-396 (1985); and references sited in each of the above, which are incorporated herein by reference. Such compounds are often developed with the aid of computerized molecular modeling. Peptide mimetics that are structurally similar to useful peptides of the present disclosure may be used to produce an equivalent effect and are therefore envisioned to be part of the present disclosure.

A “polypeptide mutant” or “mutein” refers to a polypeptide whose sequence contains an insertion, duplication, deletion, rearrangement or substitution of one or more amino acids compared to the amino acid sequence of a native or wild-type protein. A mutein may have one or more amino acid point substitutions, in which a single amino acid at a position has been changed to another amino acid, one or more insertions and/or deletions, in which one or more amino acids are inserted or deleted, respectively, in the sequence of the naturally-occurring protein, and/or truncations of the amino acid sequence at either or both the amino or carboxy termini. A mutein may have the same but preferably has a different biological activity compared to the naturally-occurring protein.

A mutein has at least 85% overall sequence homology to its wild-type counterpart. Even more preferred are muteins having at least 90% overall sequence homology to the wild-type protein.

In an even more preferred aspect, a mutein exhibits at least 95% sequence identity, even more preferably 98%, even more preferably 99% and even more preferably 99.9% overall sequence identity.

Sequence homology may be measured by any common sequence analysis algorithm, such as Gap or Bestfit.

Amino acid substitutions can include those which: (1) reduce susceptibility to proteolysis, (2) reduce susceptibility to oxidation, (3) alter binding affinity for forming protein complexes, (4) alter binding affinity or enzymatic activity, and (5) confer or modify other physicochemical or functional properties of such analogs.

As used herein, the twenty conventional amino acids and their abbreviations follow conventional usage. See Immunology—A Synthesis (Golub and Gren eds., Sinauer Associates, Sunderland, Mass., 2^{nd} ed. 1991), which is incorporated herein by reference. Stereoisomers (e.g., D-amino acids) of the twenty conventional amino acids, unnatural amino acids such as α-, α-disubstituted amino acids, N-alkyl amino acids, and other unconventional amino acids may also be suitable components for polypeptides of the present disclosure. Examples of unconventional amino acids include: 4-hydroxyproline, ≡-carboxyglutamate, ε-N,N,N-trimethyllysine, ε-N-acetyllysine, O-phosphoserine, N-acetylserine, N-formylmethionine, 3-methylhistidine, 5-hydroxylysine, N-methylarginine, and other similar amino acids and imino acids (e.g., 4-hydroxyproline). In the polypeptide notation used herein, the left-hand end corresponds to the amino terminal end and the right-hand end corresponds to the carboxy-terminal end, in accordance with standard usage and convention.

A protein has “homology” or is “homologous” to a second protein if the nucleic acid sequence that encodes the protein has a similar sequence to the nucleic acid sequence that encodes the second protein. Alternatively, a protein has homology to a second protein if the two proteins have “similar” amino acid sequences. (Thus, the term “homologous proteins” is defined to mean that the two proteins have similar amino acid sequences.) As used herein, homology between two regions of amino acid sequence (especially with respect to predicted structural similarities) is interpreted as implying similarity in function.

When “homologous” is used in reference to proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art. See, e.g., Pearson, 1994, Methods Mol. Biol. 24:307-31 and 25:365-89 (herein incorporated by reference).

The following six groups each contain amino acids that are conservative substitutions for one another: 1) Serine (S), Threonine (T); 2) Aspartic Acid (D), Glutamic Acid (E); 3) Asparagine (N), Glutamine (Q); 4) Arginine (R), Lysine (K); 5) Isoleucine (I), Leucine (L), Methionine (M), Alanine (A), Valine (V), and 6) Phenylalanine (F), Tyrosine (Y), Tryptophan (W).

Sequence homology for polypeptides, which is also referred to as percent sequence identity, is typically measured using sequence analysis software. See, e.g., the Sequence Analysis Software Package of the Genetics Computer Group (GCG), University of Wisconsin Biotechnology Center, 910 University Avenue, Madison, Wis. 53705. Protein analysis software matches similar sequences using a measure of homology assigned to various substitutions, deletions and other modifications, including conservative amino acid substitutions. For instance, GCG contains programs such as “Gap” and “Bestfit” which can be used with default parameters to determine sequence homology or sequence identity between closely related polypeptides, such as homologous polypeptides from different species of organisms or between a wild-type protein and a mutein thereof. See, e.g., GCG Version 6.1.

An algorithm that can be used when comparing a particular polypeptide sequence to a database containing a large number of sequences from different organisms is the computer program BLAST (Altschul et al., J. Mol. Biol. 215:403-410 (1990); Gish and States, Nature Genet. 3:266-272 (1993); Madden et al., Meth. Enzymol. 266:131-141 (1996); Altschul et al., Nucleic Acids Res. 25:3389-3402 (1997); Zhang and Madden, Genome Res. 7:649-656 (1997)), especially blastp or tblastn (Altschul et al., Nucleic Acids Res. 25:3389-3402 (1997)).

The length of polypeptide sequences compared for homology will generally be at least about 16 amino acid residues, usually at least about 20 residues, more usually at least about 24 residues, typically at least about 28 residues, and preferably more than about 35 residues. When searching a database containing sequences from a large number of different organisms, it can be preferable to compare amino acid sequences. Database searching using amino acid sequences can be measured by algorithms other than blastp known in the art. For instance, polypeptide sequences can be compared using FASTA, a program in GCG Version 6.1. FASTA provides alignments and percent sequence identity of the regions of the best overlap between the query and search sequences. Pearson, Methods Enzymol. 183:63-98 (1990) (incorporated by reference herein). For example, percent sequence identity between amino acid sequences can be determined using FASTA with its default parameters (a word size of 2 and the PAM250 scoring matrix), as provided in GCG Version 6.1, herein incorporated by reference.

“Specific binding” refers to the ability of two molecules to bind to each other in preference to binding to other molecules in the environment. Typically, “specific binding” discriminates over adventitious binding in a reaction by at least two-fold, more typically by at least 10-fold, often at least 100-fold. Typically, the affinity or avidity of a specific binding reaction, as quantified by a dissociation constant, is about 10⁻⁷ M or stronger (e.g., about 10⁻⁸ M, 10⁻⁹ M or even stronger).

“Percent dry cell weight” refers to a measurement of hydrocarbon production obtained as follows: a defined volume of culture is centrifuged to pellet the cells. Cells are washed then dewetted by at least one cycle of microcentrifugation and aspiration. Cell pellets are lyophilized overnight, and the tube containing the dry cell mass is weighed again such that the mass of the cell pellet can be calculated within ±0.1 mg. At the same time cells are processed for dry cell weight determination, a second sample of the culture in question is harvested, washed, and dewetted. The resulting cell pellet, corresponding to 1-3 mg of dry cell weight, is then extracted by vortexing in approximately 1 ml acetone plus butylated hydroxytolune (BHT) as antioxidant and an internal standard, e.g., n-eicosane. Cell debris is then pelleted by centrifugation and the supernatant (extractant) is taken for analysis by GC. For accurate quantitation of n-alkanes, flame ionization detection (FID) is used as opposed to MS total ion count. n-Alkane concentrations in the biological extracts are calculated using calibration relationships between GC-FID peak area and known concentrations of authentic n-alkane standards. Knowing the volume of the extractant, the resulting concentrations of the n-alkane species in the extractant, and the dry cell weight of the cell pellet extracted, the percentage of dry cell weight that comprised n-alkanes can be determined.

The term “region” as used herein refers to a physically contiguous portion of the primary structure of a biomolecule. In the case of proteins, a region is defined by a contiguous portion of the amino acid sequence of that protein.

The term “domain” as used herein refers to a structure of a biomolecule that contributes to a known or suspected function of the biomolecule. Domains may be co-extensive with regions or portions thereof; domains may also include distinct, non-contiguous regions of a biomolecule. Examples of protein domains include, but are not limited to, an Ig domain, an extracellular domain, a transmembrane domain, and a cytoplasmic domain.

As used herein, the term “molecule” means any compound, including, but not limited to, a small molecule, peptide, protein, sugar, nucleotide, nucleic acid, lipid, etc., and such a compound can be natural or synthetic.

“Carbon-based Products of Interest” include alcohols such as ethanol, propanol, isopropanol, butanol, fatty alcohols, fatty acid esters, wax esters; hydrocarbons and alkanes such as propane, octane, diesel, Jet Propellant 8 (JP8); polymers such as terephthalate, 1,3-propanediol, 1,4-butanediol, polyols, Polyhydroxyalkanoates (PHA), poly-beta-hydroxybutyrate (PHB), acrylate, adipic acid, 8-caprolactone, isoprene, caprolactam, rubber; commodity chemicals such as lactate, docosahexaenoic acid (DHA), 3-hydroxypropionate, γ-valerolactone, lysine, serine, aspartate, aspartic acid, sorbitol, ascorbate, ascorbic acid, isopentenol, lanosterol, omega-3 DHA, lycopene, itaconate, 1,3-butadiene, ethylene, propylene, succinate, citrate, citric acid, glutamate, malate, 3-hydroxypropionic acid (HPA), lactic acid, THF, gamma butyrolactone, pyrrolidones, hydroxybutyrate, glutamic acid, levulinic acid, acrylic acid, malonic acid; specialty chemicals such as carotenoids, isoprenoids, itaconic acid; pharmaceuticals and pharmaceutical intermediates such as 7-aminodeacetoxycephalosporanic acid (7-ADCA)/cephalosporin, erythromycin, polyketides, statins, paclitaxel, docetaxel, terpenes, peptides, steroids, omega fatty acids and other such suitable products of interest. Such products are useful in the context of biofuels, industrial and specialty chemicals, as intermediates used to make additional products, such as nutritional supplements, neutraceuticals, polymers, paraffin replacements, personal care products and pharmaceuticals.

Biofuel: A biofuel refers to any fuel that derives from a biological source. Biofuel can refer to one or more hydrocarbons, one or more alcohols, one or more fatty esters or a mixture thereof.

Hydrocarbon: The term generally refers to a chemical compound that consists of the elements carbon (C), hydrogen (H) and optionally oxygen (O). There are essentially three types of hydrocarbons, e.g., aromatic hydrocarbons, saturated hydrocarbons and unsaturated hydrocarbons such as alkenes, alkynes, and dienes. The term also includes fuels, biofuels, plastics, waxes, solvents and oils. Hydrocarbons encompass biofuels, as well as plastics, waxes, solvents and oils.

Throughout this specification and claims, the word “comprise” or variations such as “comprises” or “comprising”, will be understood to imply the inclusion of a stated integer or group of integers but not the exclusion of any other integer or group of integers.

In another aspect, the nucleic acid molecule of the present disclosure encodes a polypeptide having the amino acid sequence of any of the protein sequences provided in the SEQ ID NOs of the sequence listing. In some aspects, the nucleic acid molecule of the present disclosure encodes a polypeptide sequence of at least 50%, 60, 70%, 80%, 85%, 90% or 95% identity to one of the protein sequences shown in the SEQ ID NOs in the sequence listing and the identity can even more preferably be 96%, 97%, 98%, 99%, 99.9% or even higher.

The present disclosure also provides nucleic acid molecules that hybridize under stringent conditions to the above-described nucleic acid molecules. As defined above, and as is well known in the art, stringent hybridizations are performed at about 25° C. below the thermal melting point (Tₘ) for the specific DNA hybrid under a particular set of conditions, where the Tₘ is the temperature at which 50% of the target sequence hybridizes to a perfectly matched probe. Stringent washing is performed at temperatures about 5° C. lower than the Tₘ for the specific DNA hybrid under a particular set of conditions.

Nucleic acid molecules comprising a fragment of any one of the above-described nucleic acid sequences are also provided. These fragments preferably contain at least 20 contiguous nucleotides. More preferably the fragments of the nucleic acid sequences contain at least 25, 30, 35, 40, 45, 50, 60, 70, 80, 90, 100 or even more contiguous nucleotides.

The nucleic acid sequence fragments of the present disclosure display utility in a variety of systems and methods. For example, the fragments may be used as probes in various hybridization techniques. Depending on the method, the target nucleic acid sequences may be either DNA or RNA. The target nucleic acid sequences may be fractionated (e.g., by gel electrophoresis) prior to the hybridization, or the hybridization may be performed on samples in situ. One of skill in the art will appreciate that nucleic acid probes of known sequence find utility in determining chromosomal structure (e.g., by Southern blotting) and in measuring gene expression (e.g., by Northern blotting). In such experiments, the sequence fragments are preferably detectably labeled, so that their specific hydridization to target sequences can be detected and optionally quantified. One of skill in the art will appreciate that the nucleic acid fragments of the present disclosure may be used in a wide variety of blotting techniques not specifically described herein.

It should also be appreciated that the nucleic acid sequence fragments disclosed herein also find utility as probes when immobilized on microarrays. Methods for creating microarrays by deposition and fixation of nucleic acids onto support substrates are well known in the art. Reviewed in DNA Microarrays: A Practical Approach (Practical Approach Series), Schena (ed.), Oxford University Press (1999) (ISBN: 0199637768); Nature Genet. 21(1)(suppl):1-60 (1999); Microarray Biochip: Tools and Technology, Schena (ed.), Eaton Publishing Company/BioTechniques Books Division (2000) (ISBN: 1881299376), the disclosures of which are incorporated herein by reference in their entireties. Analysis of, for example, gene expression using microarrays comprising nucleic acid sequence fragments, such as the nucleic acid sequence fragments disclosed herein, is a well-established utility for sequence fragments in the field of cell and molecular biology. Other uses for sequence fragments immobilized on microarrays are described in Gerhold et al., Trends Biochem. Sci. 24:168-173 (1999) and Zweiger, Trends Biotechnol. 17:429-436 (1999); DNA Microarrays: A Practical Approach (Practical Approach Series), Schena (ed.), Oxford University Press (1999) (ISBN: 0199637768); Nature Genet. 21(1)(suppl):1-60 (1999); Microarray Biochip: Tools and Technology, Schena (ed.), Eaton Publishing Company/BioTechniques Books Division (2000) (ISBN: 1881299376), the disclosure of each of which is incorporated herein by reference in its entirety.

As is well known in the art, enzyme activities can be measured in various ways. For example, the pyrophosphorolysis of OMP may be followed spectroscopically (Grubmeyer et al., (1993) J. Biol. Chem. 268:20299-20304). Alternatively, the activity of the enzyme can be followed using chromatographic techniques, such as by high performance liquid chromatography (Chung and Sloan, (1986) J. Chromatogr. 371:71-81). As another alternative the activity can be indirectly measured by determining the levels of product made from the enzyme activity. These levels can be measured with techniques including aqueous chloroform/methanol extraction as known and described in the art (Cf M. Kates (1986) Techniques of Lipidology; Isolation, analysis and identification of Lipids. Elsevier Science Publishers, New York (ISBN: 0444807322)). More modern techniques include using gas chromatography linked to mass spectrometry (Niessen, W. M. A. (2001). Current practice of gas chromatography—mass spectrometry. New York, N.Y: Marcel Dekker. (ISBN: 0824704738)). Additional modern techniques for identification of recombinant protein activity and products including liquid chromatography-mass spectrometry (LCMS), high performance liquid chromatography (HPLC), capillary electrophoresis, Matrix-Assisted Laser Desorption Ionization time of flight-mass spectrometry (MALDI-TOF MS), nuclear magnetic resonance (NMR), near-infrared (NIR) spectroscopy, viscometry (Knothe, G (1997) Am. Chem. Soc. Symp. Series, 666: 172-208), titration for determining free fatty acids (Komers (1997) Fett/Lipid, 99(2): 52-54), enzymatic methods (Bailer (1991) Fresenius J. Anal. Chem. 340(3): 186), physical property-based methods, wet chemical methods, etc. can be used to analyze the levels and the identity of the product produced by the organisms of the present disclosure. Other methods and techniques may also be suitable for the measurement of enzyme activity, as would be known by one of skill in the art.

Also provided by the present disclosure are vectors, including expression vectors, which comprise the above nucleic acid molecules of the present disclosure, as described further herein. In a first aspect, the vectors include the isolated nucleic acid molecules described above. In an alternative aspect, the vectors of the present disclosure include the above-described nucleic acid molecules operably linked to one or more expression control sequences. The vectors of the instant disclosure may thus be used to express a katE and/or katG polypeptide contributing to H₂O₂ resistance in a host cell. In another aspect of the present disclosure, host cells transformed with the nucleic acid molecules or vectors of the present disclosure, and descendants thereof, are provided. In some aspects of the present disclosure, these cells carry the nucleic acid sequences of the present disclosure on vectors, which may but need not be freely replicating vectors. In other aspects of the present disclosure, the nucleic acids have been integrated into the genome of the host cells.

In a preferred aspect, the engineered host cell comprises one or more recombinant katE or katG encoding nucleic acids which express katE (catalase) or katG (catalase/peroxidase) in the host cell.

The term “microorganism” includes prokaryotic and eukaryotic microbial species from the Domains Archaea, Bacteria and Eucarya, the latter including yeast and filamentous fungi, protozoa, algae, or higher Protista. The terms “microbial cells” and “microbes” are used interchangeably with the term microorganism.

A variety of host organisms can be transformed to produce a product of interest. Photoautotrophic organisms include eukaryotic plants and algae, as well as prokaryotic cyanobacteria, green-sulfur bacteria, green non-sulfur bacteria, purple sulfur bacteria, and purple non-sulfur bacteria.

Extremophiles are also contemplated as suitable organisms. Such organisms withstand various environmental parameters such as temperature, radiation, pressure, gravity, vacuum, desiccation, salinity, pH, oxygen tension, and chemicals. They include hyperthermophiles, which grow at or above 80° C. such as Pyrolobus fumarii; thermophiles, which grow between 60-80° C. such as Synechococcus lividis; mesophiles, which grow between 15-60° C. and psychrophiles, which grow at or below 15° C. such as Psychrobacter and some insects. Radiation tolerant organisms include Deinococcus radiodurans. Pressure-tolerant organisms include piezophiles, which tolerate pressure of 130 MPa. Weight-tolerant organisms include barophiles. Hypergravity (e.g., >1 g) and hypogravity (e.g., <1 g) tolerant organisms are also contemplated. Vacuum tolerant organisms include tardigrades, insects, microbes and seeds. Dessicant tolerant and anhydrobiotic organisms include xerophiles such as Artemia salina; nematodes, microbes, fungi and lichens. Salt-tolerant organisms include halophiles (e.g., 2-5 M NaCl) Halobacteriacea and Dunaliella salina. pH-tolerant organisms include alkaliphiles such as Natronobacterium, Bacillus firmus OF4, Spirulina spp. (e.g., pH>9) and acidophiles such as Cyanidium caldarium, Ferroplasma sp. (e.g., low pH). Anaerobes, which cannot tolerate O₂ such as Methanococcus jannaschii; microaerophils, which tolerate some O₂ such as Clostridium and aerobes, which require O₂ are also contemplated. Gas-tolerant organisms, which tolerate pure CO₂ include Cyanidium caldarium and metal tolerant organisms include metalotolerants such as Ferroplasma acidarmanus (e.g., Cu, As, Cd, Zn), Ralstonia sp. CH34 (e.g., Zn, Co, Cd, Hg, Pb). Gross, Michael. Life on the Edge: Amazing Creatures Thriving in Extreme Environments. New YorK: Plenum (1998) and Seckbach, J. “Search for Life in the Universe with Terrestrial Microbes Which Thrive Under Extreme Conditions.” In Cristiano Batalli Cosmovici, Stuart Bowyer, and Dan Wertheimer, eds., Astronomical and Biochemical Origins and the Search for Life in the Universe, p. 511. Milan: Editrice Compositori (1997).

Plants include but are not limited to the following genera: Arabidopsis, Beta, Glycine, Jatropha, Miscanthus, Panicum, Phalaris, Populus, Saccharum, Salix, Simmondsia and Zea.

Algae and cyanobacteria include but are not limited to the following genera: Acanthoceras, Acanthococcus, Acaryochloris, Achnanthes, Achnanthidium, Actinastrum, Actinochloris, Actinocyclus, Actinotaenium, Amphichrysis, Amphidinium, Amphikrikos, Amphipleura, Amphiprora, Amphithrix, Amphora, Anabaena, Anabaenopsis, Aneumastus, Ankistrodesmus, Ankyra, Anomoeoneis, Apatococcus, Aphanizomenon, Aphanocapsa, Aphanochaete, Aphanothece, Apiocystis, Apistonema, Arthrodesmus, Artherospira, Ascochloris, Asterionella, Asterococcus, Audouinella, Aulacoseira, Bacillaria, Balbiania, Bambusina, Bangia, Basichlamys, Batrachospermum, Binuclearia, Bitrichia, Blidingia, Botrdiopsis, Botrydium, Botryococcus, Botryosphaerella, Brachiomonas, Brachysira, Brachytrichia, Brebissonia, Bulbochaete, Bumilleria, Bumilleriopsis, Caloneis, Calothrix, Campylodiscus, Capsosiphon, Carteria, Catena, Cavinula, Centritractus, Centronella, Ceratium, Chaetoceros, Chaetochloris, Chaetomorpha, Chaetonella, Chaetonema, Chaetopeltis, Chaetophora, Chaetosphaeridium, Chamaesiphon, Chara, Characiochloris, Characiopsis, Characium, Charales, Chilomonas, Chlainomonas, Chlamydoblepharis, Chlamydocapsa, Chlamydomonas, Chlamydomonopsis, Chlamydomyxa, Chlamydonephris, Chlorangiella, Chlorangiopsis, Chlorella, Chlorobotrys, Chlorobrachis, Chlorochytrium, Chlorococcum, Chlorogloea, Chlorogloeopsis, Chlorogonium, Chlorolobion, Chloromonas, Chlorophysema, Chlorophyta, Chlorosaccus, Chlorosarcina, Choricystis, Chromophyton, Chromulina, Chroococcidiopsis, Chroococcus, Chroodactylon, Chroomonas, Chroothece, Chrysamoeba, Chrysapsis, Chrysidiastrum, Chrysocapsa, Chrysocapsella, Chrysochaete, Chrysochromulina, Chrysococcus, Chrysocrinus, Chrysolepidomonas, Chrysolykos, Chrysonebula, Chrysophyta, Chrysopyxis, Chrysosaccus, Chrysophaerella, Chrysostephanosphaera, Clodophora, Clastidium, Closteriopsis, Closterium, Coccomyxa, Cocconeis, Coelastrella, Coelastrum, Coelosphaerium, Coenochloris, Coenococcus, Coenocystis, Colacium, Coleochaete, Collodictyon, Compsogonopsis, Compsopogon, Conjugatophyta, Conochaete, Coronastrum, Cosmarium, Cosmioneis, Cosmocladium, Crateriportula, Craticula, Crinalium, Crucigenia, Crucigeniella, Cryptoaulax, Cryptomonas, Cryptophyta, Ctenophora, Cyanodictyon, Cyanonephron, Cyanophora, Cyanophyta, Cyanothece, Cyanothomonas, Cyclonexis, Cyclostephanos, Cyclotella, Cylindrocapsa, Cylindrocystis, Cylindrospermum, Cylindrotheca, Cymatopleura, Cymbella, Cymbellonitzschia, Cystodinium Dactylococcopsis, Debarya, Denticula, Dermatochrysis, Dermocarpa, Dermocarpella, Desmatractum, Desmidium, Desmococcus, Desmonema, Desmosiphon, Diacanthos, Diacronema, Diadesmis, Diatoma, Diatomella, Dicellula, Dichothrix, Dichotomococcus, Dicranochaete, Dictyochloris, Dictyococcus, Dictyosphaerium, Didymocystis, Didymogenes, Didymosphenia, Dilabifilum, Dimorphococcus, Dinobryon, Dinococcus, Diplochloris, Diploneis, Diplostauron, Distrionella, Docidium, Draparnaldia, Dunaliella, Dysmorphococcus, Ecballocystis, Elakatothrix, Ellerbeckia, Encyonema, Enteromorpha, Entocladia, Entomoneis, Entophysalis, Epichrysis, Epipyxis, Epithemia, Eremosphaera, Euastropsis, Euastrum, Eucapsis, Eucocconeis, Eudorina, Euglena, Euglenophyta, Eunotia, Eustigmatophyta, Eutreptia, Fallacia, Fischerella, Fragilaria, Fragilariforma, Franceia, Frustulia, Curcilla, Geminella, Genicularia, Glaucocystis, Glaucophyta, Glenodiniopsis, Glenodinium, Gloeocapsa, Gloeochaete, Gloeochrysis, Gloeococcus, Gloeocystis, Gloeodendron, Gloeomonas, Gloeoplax, Gloeothece, Gloeotila, Gloeotrichia, Gloiodictyon, Golenkinia, Golenkiniopsis, Gomontia, Gomphocymbella, Gomphonema, Gomphosphaeria, Gonatozygon, Gongrosia, Gongrosira, Goniochloris, Gonium, Gonyostomum, Granulochloris, Granulocystopsis, Groenbladia, Gymnodinium, Gymnozyga, Gyrosigma, Haematococcus, Hafniomonas, Hallassia, Hammatoidea, Hannaea, Hantzschia, Hapalosiphon, Haplotaenium, Haptophyta, Haslea, Hemidinium, Hemitoma, Heribaudiella, Heteromastix, Heterothrix, Hibberdia, Hildenbrandia, Hillea, Holopedium, Homoeothrix, Hormanthonema, Hormotila, Hyalobrachion, Hyalocardium, Hyalodiscus, Hyalogonium, Hyalotheca, Hydrianum, Hydrococcus, Hydrocoleum, Hydrocoryne, Hydrodictyon, Hydrosera, Hydrurus, Hyella, Hymenomonas, Isthmochloron, Johannesbaptistia, Juranyiella, Karayevia, Kathablepharis, Katodinium, Kephyrion, Keratococcus, Kirchneriella, Klebsormidium, Kolbesia, Koliella, Komarekia, Korshikoviella, Kraskella, Lagerheimia, Lagynion, Lamprothamnium, Lemanea, Lepocinclis, Leptosira, Lobococcus, Lobocystis, Lobomonas, Luticola, Lyngbya, Malleochloris, Mallomonas, Mantoniella, Marssoniella, Martyana, Mastigocoleus, Gastogloia, Melosira, Merismopedia, Mesostigma, Mesotaenium, Micractinium, Micrasterias, Microchaete, Microcoleus, Microcystis, Microglena, Micromonas, Microspora, Microthamnion, Mischococcus, Monochrysis, Monodus, Monomastix, Monoraphidium, Monostroma, Mougeotia, Mougeotiopsis, Myochloris, Myromecia, Myxosarcina, Naegeliella, Nannochloris, Nautococcus, Navicula, Neglectella, Neidium, Nephroclamys, Nephrocytium, Nephrodiella, Nephroselmis, Netrium, Nitella, Nitellopsis, Nitzschia, Nodularia, Nostoc, Ochromonas, Oedogonium, Oligochaetophora, Onychonema, Oocardium, Oocystis, Opephora, Ophiocytium, Orthoseira, Oscillatoria, Oxyneis, Pachycladella, Palmella, Palmodictyon, Pnadorina, Pannus, Paralia, Pascherina, Paulschulzia, Pediastrum, Pedinella, Pedinomonas, Pedinopera, Pelagodictyon, Penium, Peranema, Peridiniopsis, Peridinium, Peronia, Petroneis, Phacotus, Phacus, Phaeaster, Phaeodermatium, Phaeophyta, Phaeosphaera, Phaeothamnion, Phormidium, Phycopeltis, Phyllariochloris, Phyllocardium, Phyllomitas, Pinnularia, Pitophora, Placoneis, Planctonema, Planktosphaeria, Planothidium, Plectonema, Pleodorina, Pleurastrum, Pleurocapsa, Pleurocladia, Pleurodiscus, Pleurosigma, Pleurosira, Pleurotaenium, Pocillomonas, Podohedra, Polyblepharides, Polychaetophora, Polyedriella, Polyedriopsis, Polygoniochloris, Polyepidomonas, Polytaenia, Polytoma, Polytomella, Porphyridium, Posteriochromonas, Prasinochloris, Prasinocladus, Prasinophyta, Prasiola, Prochlorphyta, Prochlorothrix, Protoderma, Protosiphon, Provasoliella, Prymnesium, Psammodictyon, Psammothidium, Pseudanabaena, Pseudenoclonium, Psuedocarteria, Pseudochate, Pseudocharacium, Pseudococcomyxa, Pseudodictyosphaerium, Pseudokephyrion, Pseudoncobyrsa, Pseudoquadrigula, Pseudosphaerocystis, Pseudostaurastrum, Pseudostaurosira, Pseudotetrastrum, Pteromonas, Punctastruata, Pyramichlamys, Pyramimonas, Pyrrophyta, Quadrichloris, Quadricoccus, Quadrigula, Radiococcus, Radiofilum, Raphidiopsis, Raphidocelis, Raphidonema, Raphidophyta, Peimeria, Rhabdoderma, Rhabdomonas, Rhizoclonium, Rhodomonas, Rhodophyta, Rhoicosphenia, Rhopalodia, Rivularia, Rosenvingiella, Rossithidium, Roya, Scenedesmus, Scherffelia, Schizochlamydella, Schizochlamys, Schizomeris, Schizothrix, Schroederia, Scolioneis, Scotiella, Scotiellopsis, Scourfieldia, Scytonema, Selenastrum, Selenochloris, Sellaphora, Semiorbis, Siderocelis, Diderocystopsis, Dimonsenia, Siphononema, Sirocladium, Sirogonium, Skeletonema, Sorastrum, Spermatozopsis, Sphaerellocystis, Sphaerellopsis, Sphaerodinium, Sphaeroplea, Sphaerozosma, Spiniferomonas, Spirogyra, Spirotaenia, Spirulina, Spondylomorum, Spondylosium, Sporotetras, Spumella, Staurastrum, Stauerodesmus, Stauroneis, Staurosira, Staurosirella, Stenopterobia, Stephanocostis, Stephanodiscus, Stephanoporos, Stephanosphaera, Stichococcus, Stichogloea, Stigeoclonium, Stigonema, Stipitococcus, Stokesiella, Strombomonas, Stylochrysalis, Stylodinium, Styloyxis, Stylosphaeridium, Surirella, Sykidion, Symploca, Synechococcus, Synechocystis, Synedra, Synochromonas, Synura, Tabellaria, Tabularia, Teilingia, Temnogametum, Tetmemorus, Tetrachlorella, Tetracyclus, Tetradesmus, Tetraedriella, Tetraedron, Tetraselmis, Tetraspora, Tetrastrum, Thalassiosira, Thamniochaete, Thorakochloris, Thorea, Tolypella, Tolypothrix, Trachelomonas, Trachydiscus, Trebouxia, Trentepholia, Treubaria, Tribonema, Trichodesmium, Trichodiscus, Trochiscia, Tryblionella, Ulothrix, Uroglena, Uronema, Urosolenia, Urospora, Uva, Vacuolaria, Vaucheria, Volvox, Volvulina, Westella, Woloszynskia, Xanthidium, Xanthophyta, Xenococcus, Zygnema, Zygnemopsis, and Zygonium. A partial list of cyanobacteria that can be engineered to express the recombinant described herein include members of the genus Chamaesiphon, Chroococcus, Cyanobacterium, Cyanobium, Cyanothece, Dactylococcopsis, Gloeobacter, Gloeocapsa, Gloeothece, Microcystis, Prochlorococcus, Prochloron, Synechococcus, Synechocystis, Cyanocystis, Dermocarpella, Stanieria, Xenococcus, Chroococcidiopsis, Myxosarcina, Arthrospira, Borzia, Crinalium, Geitlerinemia, Leptolyngbya, Limnothrix, Lyngbya, Microcoleus, Oscillatoria, Planktothrix, Prochiorothrix, Pseudanabaena, Spirulina, Starria, Symploca, Trichodesmium, Tychonema, Anabaena, Anabaenopsis, Aphanizomenon, Cyanospira, Cylindrospermopsis, Cylindrospermum, Nodularia, Nostoc, Scylonema, Calothrix, Rivularia, Tolypothrix, Chlorogloeopsis, Fischerella, Geitieria, Iyengariella, Nostochopsis, Stigonema and Thermosynechococcus.

Green non-sulfur bacteria include but are not limited to the following genera: Chloroflexus, Chloronema, Oscillochloris, Heliothrix, Herpetosiphon, Roseiflexus, and Thermomicrobium.

Green sulfur bacteria include but are not limited to the following genera: Chlorobium, Clathrochloris, and Prosthecochloris.

Purple sulfur bacteria include but are not limited to the following genera: Allochromatium, Chromatium, Halochromatium, Isochromatium, Marichromatium, Rhodovulum, Thermochromatium, Thiocapsa, Thiorhodococcus, and Thiocystis,

Purple non-sulfur bacteria include but are not limited to the following genera: Phaeospirillum, Rhodobaca, Rhodobacter, Rhodomicrobium, Rhodopila, Rhodopseudomonas, Rhodothalassium, Rhodospirillum, Rodovibrio, and Roseospira.

Aerobic chemolithotrophic bacteria include but are not limited to nitrifying bacteria such as Nitrobacteraceae sp., Nitrobacter sp., Nitrospina sp., Nitrococcus sp., Nitrospira sp., Nitrosomonas sp., Nitrosococcus sp., Nitrosospira sp., Nitrosolobus sp., Nitrosovibrio sp.; colorless sulfur bacteria such as, Thiovulum sp., Thiobacillus sp., Thiomicrospira sp., Thiosphaera sp., Thermothrix sp.; obligately chemolithotrophic hydrogen bacteria such as Hydrogenobacter sp., iron and manganese-oxidizing and/or depositing bacteria such as Siderococcus sp., and magnetotactic bacteria such as Aquaspirillum sp.

Archaeobacteria include but are not limited to methanogenic archaeobacteria such as Methanobacterium sp., Methanobrevibacter sp., Methanothermus sp., Methanococcus sp., Methanomicrobium sp., Methanospirillum sp., Methanogenium sp., Methanosarcina sp., Methanolobus sp., Methanothrix sp., Methanococcoides sp., Methanoplanus sp.; extremely thermophilic S-Metabolizers such as Thermoproteus sp., Pyrodictium sp., Sulfolobus sp., Acidianus sp. and other microorganisms such as, Bacillus subtilis, Saccharomyces cerevisiae, Streptomyces sp., Ralstonia sp., Rhodococcus sp., Corynebacteria sp., Brevibacteria sp., Mycobacteria sp., and oleaginous yeast.

Preferred organisms for the manufacture of n-alkanes according to the methods disclosed herein include: Arabidopsis thaliana, Panicum virgatum, Miscanthus giganteus, and Zea mays (plants); Botryococcus braunii, Chlamydomonas reinhardtii and Dunaliela salina (algae); Synechococcus sp PCC 7002, Synechococcus sp. PCC 7942, Synechocystis sp. PCC 6803, Thermosynechococcus elongatus BP-1 (cyanobacteria); Chlorobium tepidum (green sulfur bacteria), Chloroflexus auranticus (green non-sulfur bacteria); Chromatium tepidum and Chromatium vinosum (purple sulfur bacteria); Rhodospirillum rubrum, Rhodobacter capsulatus, and Rhodopseudomonas palusris (purple non-sulfur bacteria).

Yet other suitable organisms include synthetic cells or cells produced by synthetic genomes as described in Venter et al. US Pat. Pub. No. 2007/0264688, and cell-like systems or synthetic cells as described in Glass et al. US Pat. Pub. No. 2007/0269862.

Still, other suitable organisms include microorganisms that can be engineered to fix carbon dioxide bacteria such as Escherichia coli, Acetobacter aceti, Bacillus subtilis, yeast and fungi such as Clostridium ljungdahlii, Clostridium thermocellum, Penicillium chrysogenum, Pichia pastoris, Saccharomyces cerevisiae, Schizosaccharomyces pombe, Pseudomonas fluorescens, or Zymomonas mobilis.

A suitable organism for selecting or engineering is autotrophic fixation of CO₂ to products. This would cover photosynthesis and methanogenesis. Acetogenesis, encompassing the three types of CO₂ fixation; Calvin cycle, acetyl-CoA pathway and reductive TCA pathway is also covered. The capability to use carbon dioxide as the sole source of cell carbon (autotrophy) is found in almost all major groups of prokaryotes. The CO₂ fixation pathways differ between groups, and there is no clear distribution pattern of the four presently-known autotrophic pathways. See, e.g., Fuchs, G. 1989. Alternative pathways of autotrophic CO₂ fixation, p. 365-382. In H. G. Schlegel, and B. Bowien (ed.), Autotrophic bacteria. Springer-Verlag, Berlin, Germany. The reductive pentose phosphate cycle (Calvin-Bassham-Benson cycle) represents the CO₂ fixation pathway in almost all aerobic autotrophic bacteria, for example, the cyanobacteria.

### General Methods for Inhibiting Contamination in a Reactor System and Generating a Reactive Oxygen Species (ROS) Resistant Host Cell

Photosynthetic organisms are cultured in reactor systems. Reactor systems are sterilized using vapor hydrogen peroxide (VHP). VHP treatment requires that the air in the reactor system be dry, so that the hydrogen peroxide vapor does not condense into existing liquid in the reactors. The complete drying of the reactors, however, is impractical, and it must be assumed that there will be residual liquid hydrogen peroxide in the reactors prior to inoculation. This residual hydrogen peroxide is toxic to cells and can cause culture death. It is advantageous, therefore, to develop a cyanobacterial host that is resistant to hydrogen peroxide. Resistance due to hydrogen peroxide will allow the host to survive the initial challenge due to condensation from VHP. Resistance to hydrogen peroxide will also allow the host to selectively survive periodic hydrogen peroxide treatments aimed at eliminating contamination.

The resistance to hydrogen peroxide, a reactive oxygen species (ROS), in a cyanobacterial host can be achieved by increasing the catalase and/or peroxidase capacity of the host organism. Catalase and catalase/peroxidase (EC 1.11.1.6 and EC 1.11.1.21, respectively) catalyze the conversion of hydrogen peroxide to water and oxygen according to the following equation:

2H₂O₂→2H₂O+O₂

and are encoded by the katE (catalase) and katG (catalase/peroxidase) genes, respectively. In one embodiment, the resistance to hydrogen peroxide was increased in an ethanologenic strain of Synechococcus PCC 7002 (JCC1510) by overexpression of katG from Synechocystis sp PCC 6803.

The following examples are for illustrative purposes and are not intended to limit the scope of the disclosure.

### EXAMPLES

### Example 1

### Overexpression of katG in Synechococcus PCC 7002 in a JCC2979 Ethanologen

The catalase and/or peroxidase capacity of a host organism is increased by introducing a recombinant katG (catalase/peroxidase) or katE (catalase) gene, increasing resistance to reactive oxygen species, such as H₂O₂. In the following method, recombinant katG is introduced into an ethanologen, JCC1510, to increase its resistance to H₂O₂.

### Construction of the JCC1510 Strain

The JCC1510 ethanologen strain was constructed by standard homologous recombination techniques from a cyanobacterial strain. The starting material was wild-type Synechococcus sp. PCC7002 (JCC138), which was obtained from the Pasteur Collection or ATCC. The engineered strain comprises a Z. mobilis pyruvate decarboxylase gene (pdc) under the control of a P(nir07) promoter (SEQ ID NO: 5), and Moorella alcohol dehydrogenase gene (adh) from Moorella under the control of a lambda cro promoter (SEQ ID NO: 6) (Table 1).

TABLE 1
JCC1510 recombinant genes

| Strain | Genes |
|---|---|
| JCC1510 | Chromosome::P(nir07)-pdc-kan-P(cro)-adh |

### Construction of the JCC2979 Strain

Overexpression of katG (SEQ ID NO: 3) from Synechocystis sp PCC 6803 in Synechococcus PCC 7002 was achieved according to the following method. katG was amplified by PCR with KOD Polymerase from Synechocystis sp PCC 6803 genomic DNA using primers MC10 (SEQ ID NO: 1) and MC11 (SEQ ID NO: 2). The 2.3 kb PCR product was digested with NdeI and EcoRI and ligated into the corresponding sites of Synechococcus PCC 7002 integrative expression vector (pJB1591) (see Table 2). The promoter region containing P_{aphII} was excised with NotI and NdeI and replaced with the 600 bp Synechocystis sp PCC 6803 cpcB promoter fragment (chromosomal coordinates 728060-727470 (complement)) from pJB577 to form plasmid pJB1767 (see Table 2).

pJB1767 was integrated into the genome of a parent strain, Synechococcus PCC 7002 (JCC1510), by natural transformation and selection on 25 μg mL⁻¹ gentamycin. The resulting strain was renamed JCC2979.

Another catalase, such as katE from E. coli, can be used alternatively or in addition to katG.

TABLE 2
Generation of plasmid pJB1767 from pJB1591

| Plasmid | Plasmid Sequence Map |
|---|---|
| pJB1591 | AquI UHR\|(NotI) P_{aphII} (NdeI) - katG - (EcoRI)\|Gent^{R}\|AquI |
| | DHR\|Amp^{R}\|pUC ori |
| pJB577 | Amp^{R}\|ldhA UHR\|(NotI) P_{cpcB} (NdeI) - tesA - fadD - wax - |
| | dgat\|Spec^{R}\|pUC ori |
| pJB1767 | AquI UHR\|(NotI) P_{cpcB} (NdeI) - katG - (EcoRI)\|Gent^{R} \|AquI |
| | DHR\|Amp^{R}\|pUC |

### Example 2

### Viability and Ethanol Productivity of JCC1510 and JCC2979 Under Standard Conditions (Continuous Light)

Parent (JCC1510) and engineered (JCC2979) strains were inoculated in triplicate from single colonies in test tubes in A+ media with 10 mM urea and antibiotics and grown under standard conditions (continuous light; ˜100 μE m⁻² s⁻¹; 37° C.; 2% CO₂; 150 rpm). Tube precultures were used to inoculate 30 mL flask cultures with foam stoppers to OD₇₃₀=0.1 in JB2.1 media (Table 3) with 3 mM urea and grown under standard conditions. Samples were taken periodically to quantify growth (OD₇₃₀) and ethanol production (GC-FID). Evaporative losses were controlled by weighing flasks and adding Milli-Q H₂O as required. The cumulative ethanol production was calculated by integrating the system mass balance and solving for the production rate constant:

$$\frac{d {[ EtOH ]}_{( liq )}}{d t} = {k}_{1} - {{k}_{2} [ EtOH ]}_{( liq )}$$

$$Accumulation ( EtOH in media ) = Production Rate - Stripping Rate$$

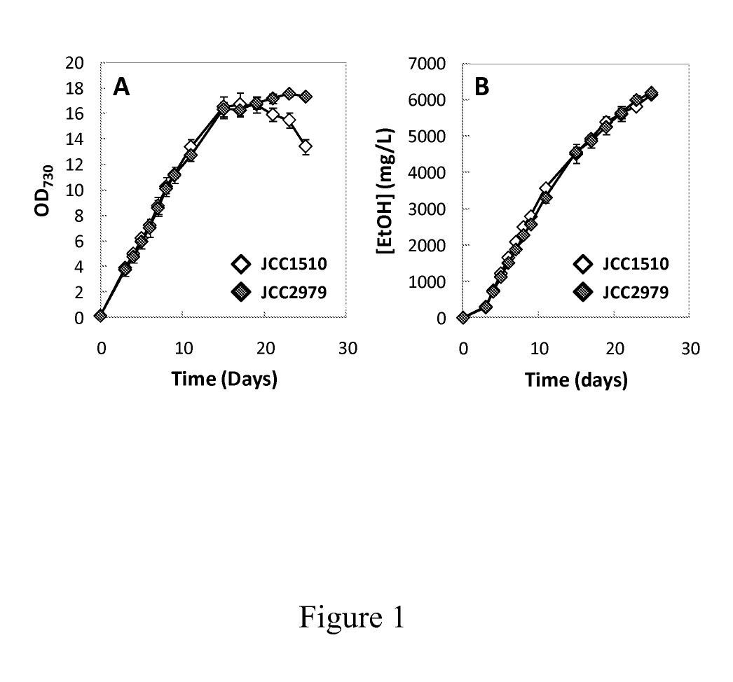

Triplicate flask cultures of JCC1510 (parent) and JCC2979 (engineered) were grown for 25 days after which growth (A) and ethanol production (B) in JCC2979 met or exceeded the levels of JCC1510 (FIG. 1).

TABLE 3
JB2.1 media

| JB2.1 media | (g/L, final) |
|---|---|
| Sodium chloride | 5.00 |
| Potassium chloride | 0.60 |
| Sodium nitrate | 3.5 |
| Magnesium sulfate heptahydrate | 5.00 |
| Potassium phosphate monobasic | 0.2 |
| EDTA, disodium salt dehydrate | 0.029 |
| Iron (III) citrate hydrate | 0.014 |
| Tris/THAM ® | 1.00 |
| Urea | 0.18 |
| Calcium chloride, anhydrous | 0.266 |
| Boric acid | 0.034 |
| Manganese chloride tetrahydrate | 0.0043 |
| Zinc chloride | 0.00032 |
| Molybdenum (VI) oxide | 0.00003 |
| Copper (II) sulfate pentahydrate | 0.000003 |
| Cobalt (II) chloride hexahydrate | 0.000012 |

### Example 3

### Viability and Ethanol Productivity of JCC1510 and JCC2979 in Natural Light Conditions

Parent (JCC1510) and engineered (JCC2979) strains were inoculated from single colonies in test tubes in A+ media with 10 mM urea and antibiotics (Spec¹⁰⁰ Kan⁵⁰ Gent²⁵) and grown under standard conditions (continuous light; ˜100 μE*m⁻²*s⁻¹; 37° C.; 2% CO₂; 150 rpm). Tube cultures were used to inoculated 30 mL flask precultures to OD₇₃₀=0.5 in JB2.1 media with 10 mM urea and antibiotics under standard conditions. Experimental cultures were inoculated from flask precultures to OD₇₃₀=0.5 in 400 mL of JB2.1 media with 10 mM urea in a FMT150 photobioreactor (Photon Systems Instruments, Czech Republic). Light was provided by a 630 nm LED array adjusted from 200-2,000 μE*m⁻²*s⁻¹ during a 12 hour light cycle according to a Gaussian distribution. CO₂ was provided by sparging 1 L min⁻¹ of 2% CO₂ in air. Each 24 hour cycle includes one 12 hour light cycle and one 12 hour dark cycle where the LED array is turned off. Growth was quantified periodically by light scattering at 730 nm. Ethanol production was measured by GC-FID.

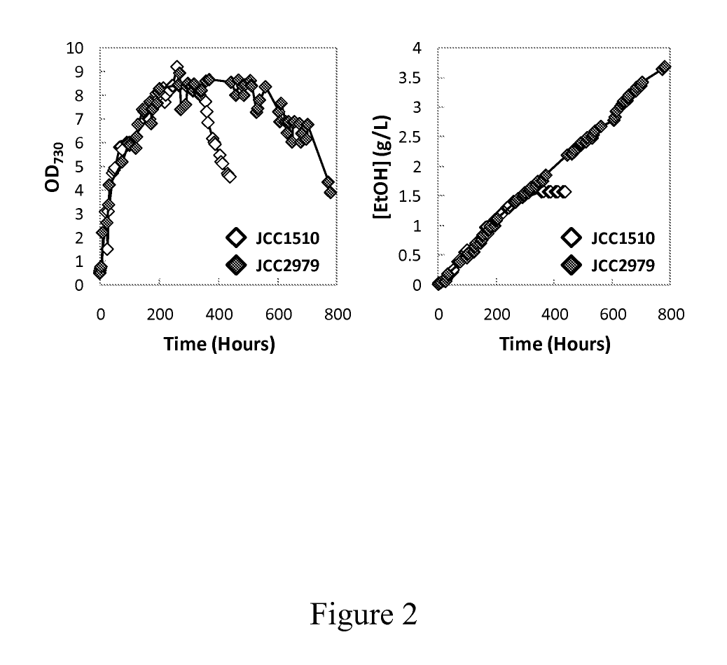

FMT150 photobioreactor cultures of JCC1510 (parent) and JCC2979 (engineered) were grown for 15-35 days after which growth (A) and ethanol production (B) in JCC2979 met or exceeded the levels of JCC1510 (FIG. 2).

### Example 4

### Viability of JCC1510 and JCC2979 Following Treatment with Hydrogen Peroxide

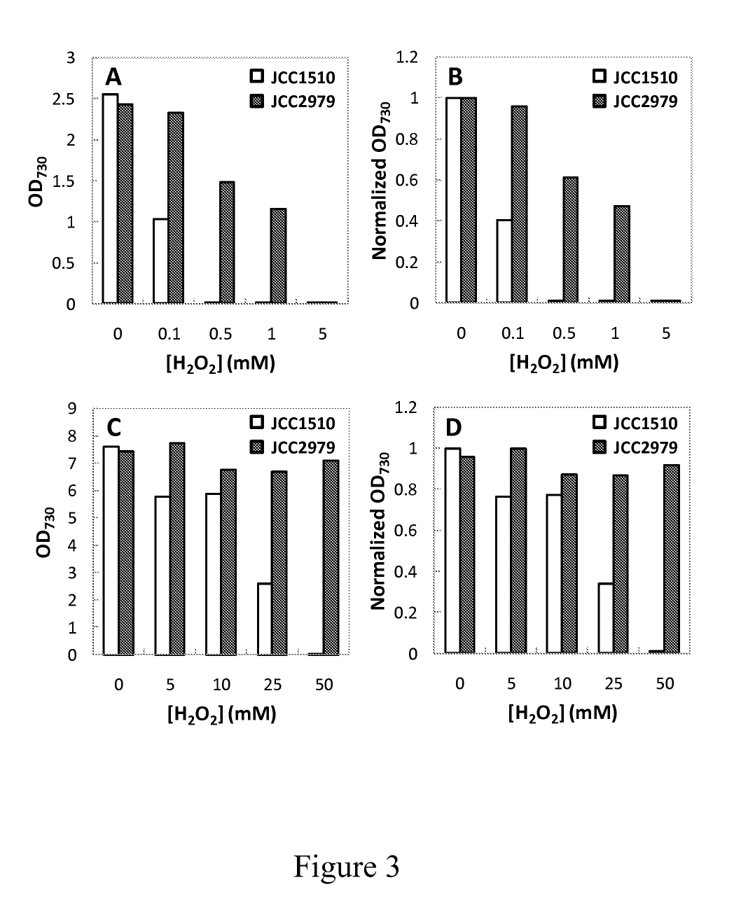

Parent (JCC1510) and engineered (JCC2979) strains were inoculated from single colonies in test tubes in A+ media with 10 mM urea and antibiotics (Spec¹⁰⁰ Kan⁵⁰ Gent²⁵) and grown under standard conditions (continuous light; ˜100 μE m⁻² s⁻¹; 37° C.; 2% CO₂; 150 rpm). Tube cultures of JCC1510 and JCC2979 were inoculated to OD₇₃₀=0.1 (FIG. 3A,B) or OD₇₃₀=3.0 (FIG. 3C,D) and challenged with selected levels of hydrogen peroxide. Bars represent absolute (FIG. 3A,C) or normalized (FIG. 3B,D) OD₇₃₀ after 24 hours. Normalized OD₇₃₀ values were calculated by dividing each measured OD₇₃₀ value after 24 hours with the highest absolute OD₇₃₀ value after 24 hours.

As shown in FIG. 3A-D, the engineered ethanologen JCC2979 strain comprising recombinant katG possesses a greater resistance to H₂O₂ than its parent ethanologen strain JCC1510.

### Example 5

### Overexpression of katG in Synechococcus PCC 7002 in a JCC3351 Ethanologen

### Construction of the JCC1581 Strain

The JCC1581 strain was constructed by standard homologous recombination techniques. The starting material was wild-type Synechococcus sp. PCC7002 (JCC138), which was obtained from the Pasteur Collection or ATCC. Gene, promoter, terminator, and marker constructs made synthetically were obtained from DNA2.0 or by PCR, oligonucleotides for PCR and sequence confirmation from IDT. DCCD (N,N′-dicyclohexylcarbodiimide), TCS (3,3′,4′,5-tetrachlorosalicylanilide), 2,4-DNP (2,4-dinitrophenol), and CCCP (carbonyl cyanide-p-trifluoromethoxyhydrazone) were obtained from Sigma.

As previously described in published PCT application WO2010/044960, a pAQ7 Δldh targeting plasmid (see Genbank # CP000957) was constructed containing the Moorella alcohol dehydrogenase gene (adh) under the control of the lambda cro promoter (SEQ ID NO: 6). This plasmid (pJB594) was naturally transformed into JCC138 (see Table 2) using a standard cyanobacterial transformation protocol, yielding strain JCC1034.

Briefly, JCC138 culture was grown to an OD730 of approximately 1.0, after which 5-10 μg of plasmid DNA (pJB594) was added to 1 ml of neat JCC138 culture. The cell-DNA mixture was incubated at 37° C. for 4 hours in the dark with gentle mixing, plated onto A+ plates, and incubated in a photoincubator (Percival) for 24 hours, at which point kanamycin was underlaid to a final concentration of 50 μg/ml. Kanamycin-resistant colonies (JCC1034) appeared after 5-8 days of further incubation under 24 hr-light conditions (˜100 μmol photons m⁻²*s⁻¹). One round of colony purification was performed on A+ plates supplemented with 50 μg/ml kanamycin. Single colonies of each of the six transformed strains (JCC1034) was grown in test-tubes for 4-8 days at 37° C. at 150 rpm in 3% CO₂-enriched air at ˜100 μmol photons m⁻²*s⁻¹ in a Multitron II (Infors) shaking photoincubator. The growth medium for liquid culture was A+ with 50 μg/ml kanamycin.

JCC1034 was then transformed with pJB1156 which introduced a two-gene operon, driven by P(nir07) (SEQ ID NO: 5), to an ectopic location on pAQ3. This operon contained Moorella alcohol dehydrogenase gene (adh) as well as the pyruvate decarboxylase gene (pdc) from Zymomonas mobilis. The resulting strain, JCC1581, therefore had two independently expressed adh transgenes and one pdc transgene. The protocol used to transform pJB1156 into JCC1034 generating JCC1581 was the same as above with the exception that the selection media is A+ containing 3 mM urea, 50 μg/ml kanamycin, and a 25 μg/ml spectinomycin underlay.

TABLE 4
Transformation of host cell with integrative plasmid
generated JCC1034 and JCC1518 strains from
wild-type Synechococcus sp. PCC7002 (JCC138).

| Strain | Host | Integrative Plasmid |
|---|---|---|
| JCC1034 | JCC138 | pJB594 (pAQ7::Δldh_kan_P(cro)_adh) |
| JCC1581 | JCC1034 | pJB1156 (pAQ3::P(nir07)_pdc_adh_spec) |

### Construction of the JCC3351 Strain

pJB1767 (Table 4) expressing katG was integrated into the genome of a parent strain, (JCC1581; described above) by natural transformation and selection on 25 n*mL⁻¹ gentamycin. The resulting strain was renamed JCC3351.

Another catalase, such as katE from E. coli, can be used alternatively or in addition to katG.

### Example 6

### Viability and Ethanol Productivity of JCC1581 and JCC3351 in Natural Light Conditions

As described above, katG was integrated into the genome of a parent strain JCC1581 to produce strain JCC3351. The effect of expression of katG on ethanol productivity was tested by culturing and comparing JCC1581 (control) and JCC3351 (katG experimental). Parent (JCC1581) and engineered (JCC3351) strains were inoculated from single colonies in test tubes in A+ media with 10 mM urea and antibiotics (Spec100 Kan50 Gent25) and grown under standard conditions (continuous light; ˜100 μE*m⁻²*s⁻¹; 37° C.; 2% CO₂; 150 rpm). Tube cultures were used to inoculated 30 mL flask precultures to OD730=0.5 in JB2.1 media with 10 mM urea and antibiotics under standard conditions. Experimental cultures were inoculated from flask precultures to OD730=0.1 in 400 mL of JB2.1 media with 3 mM urea in a FMT150 photobioreactor (Photon Systems Instruments, Czech Republic). Light was provided by a 630 nm LED array adjusted from 130-1,300 g*m⁻²*s⁻¹ during a 12 hour light cycle according to a Gaussian distribution. CO₂ was provided by sparging 1 L*min⁻¹ of 2% CO₂ in N₂. Each 24 hour cycle includes one 12 hour light cycle and one 12 hour dark cycle where the LED array is turned off. Growth was quantified periodically by light scattering at 730 nm. Ethanol production was measured by GC-FID.

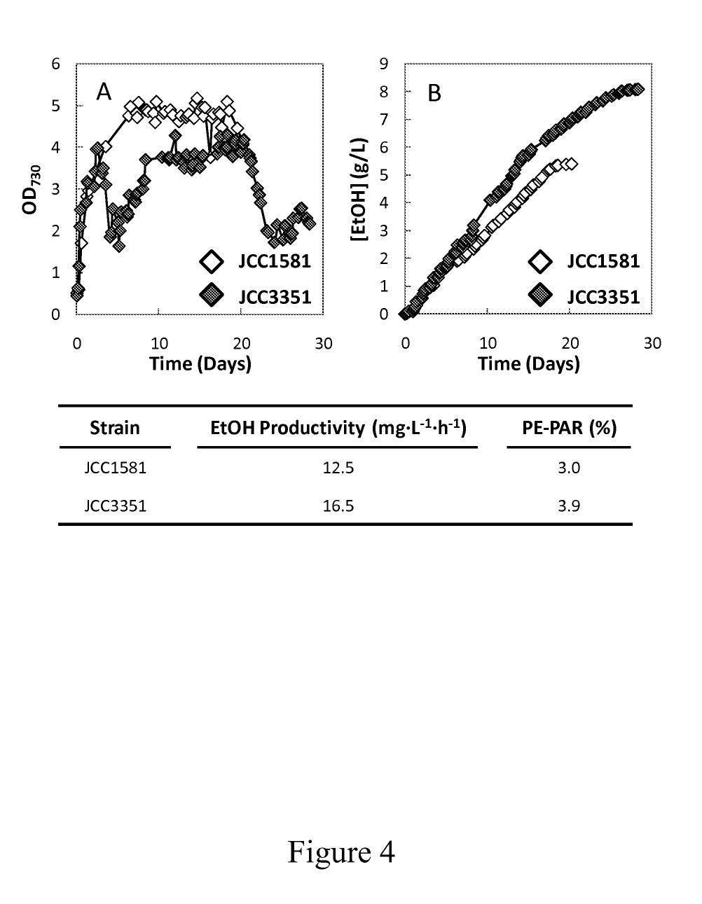

FMT150 photobioreactor cultures of JCC1581 (parent) and JCC3351 (engineered) were grown for up to 29 days. Growth (FIG. 4A) and total ethanol production (FIG. 4B) was measured over the course of the experiment from 0 to 30 days. Ethanol production in JCC3351 (expressing heterologous katG) exceeded ethanol production in JCC1581 (parent).

1. A method to produce a carbon-based product of interest, comprising culturing an engineered cyanobacterial cell in a cell culture in the presence of CO₂ and light under conditions suitable to produce a carbon-based product of interest, wherein said engineered cyanobacterial cell comprises a recombinant nucleic acid encoding an enzyme classified under an Enzyme Commission number selected from EC 1.11.1.6, EC 1.11.1.7, and EC 1.11.1.21.

2. The method of claim 1, wherein said enzyme has catalase activity.

3. The method of claim 1, wherein said enzyme has peroxidase activity.

4. The method of claim 1, wherein said enzyme is selected from KatG and KatE.

5. The method of claim 1, wherein said enzyme comprises an amino acid sequence at least 90% identical to SEQ ID NO: 3.

6. The method of claim 1, wherein said enzyme comprises an amino acid sequence at least 90% identical to SEQ ID NO: 4.

7. The method of claim 1, wherein said cell culture further comprises hydrogen peroxide.

8. The method of claim 7, wherein said hydrogen peroxide is at a concentration of 0.1 to 50 mM in said cell culture.

9. The method of claim 1, wherein said cell culture comprises a reagent to mitigate contamination of said cell culture.

10. The method of claim 1, wherein said cell culture is exposed to diurnal light conditions.

11. The method of claim 1, wherein said engineered cyanobacterial cell has the same or a higher rate of ethanol production in said cell culture than an otherwise identical cyanobacterial cell lacking said recombinant nucleic acid.

12. The method of claim 1, wherein the production rate of ethanol of a culture of said engineered cyanobacterial cells is greater than a rate selected from: 50, 100, 150, 200, 250, 300, 350, and 400 mg*L⁻¹*day⁻¹, or wherein the production rate of ethanol of a culture of said engineered byanobacterial cells is greater than a rate selected from: 13, 14, 15, and 16 mg*L⁻¹*h⁻¹, or wherein the mass of ethanol produced by a culture of said engineered cyanobacterial cells per volume of said culture is greater than an amount selected from: 1.5, 2, 2.5, 3, 3.5, 4, 4.5, 5, 5.5, 6, 6.5, 7, 7.5, and 8 g/L, or wherein said ethanol is produced during up to 30 days of culture.

13-15. (canceled)

16. The method of claim 1, wherein said cyanobacterial cell is an ethanologen, a Synechococcus species, or Synechococcus PCC 7002.

17-18. (canceled)

19. A method for conferring hydrogen peroxide resistance to a parent cyanobacterial cell, comprising:
a. transforming said parent cyanobacterial cell with a nucleic acid encoding an enzyme selected from KatG and KatE, resulting in an engineered cyanobacterial cell; and
b. culturing said engineered cyanobacterial cell in a medium comprising hydrogen peroxide.

20-26. (canceled)

27. An engineered host cell comprising a recombinant gene encoding a first enzyme classified under an Enzyme Commission number selected from EC 1.11.1.6, EC 1.11.1.7, and EC 1.11.1.21, wherein said host cell further comprises a recombinant pyruvate decarboxylase or a recombinant alcohol dehydrogenase.

28. The engineered host cell of claim 27, wherein said host cell is a cyanobacterium.

29. The engineered host cell of claim 28, wherein said cyanobacterium is Synechococcus PCC 7002.

30. The engineered host cell of claim 27, wherein said first enzyme is selected from KatG and KatE.

31. The engineered host cell of claim 27, wherein said first enzyme comprises an amino acid sequence at least 90% identical to SEQ ID NO: 3.

32. The engineered host cell of claim 27, wherein said first enzyme comprises an amino acid sequence at least 90% identical to SEQ ID NO: 4.


SEQUENCE LISTING (6 sequences)

SEQ ID NO: 1 - DNA, 37 bp, Artificial Sequence
  Description of Artificial Sequence Synthetic primer
ctaacatatgggcacccaacccgccaggaaattaaga

SEQ ID NO: 2 - DNA, 37 bp, Artificial Sequence
  Description of Artificial Sequence Synthetic primer
ctaagaattcctagcccctagggagatcaaaccggtc

SEQ ID NO: 3 - protein, 754 aa, Synechocystis sp.
MGTQPARKLRNRVFPHPHNHRKEKPMANDQVPASKCPVMHGANTTGQNGNLNWWPNALNLDILHQHDRKTNPMDDGFNYAEAFQQLDLAAVKQDLHHLMTDSQSWWPADWGHYGGLMIRMAWHAAGTYRIADGRGGAATGNQRFAPLNSWPDNVNLDKARRLLWPIKKKYGNKLSWGDLIILAGTMAYESMGLKVYGFAGGREDIWHPEKDIYWGAEKEWLASSDHRYGSEDRESLENPLAAVQMGLIYVNPEGVDGHPDPLCTAQDVRTTFARMAMNDEETVALTAGGHTVGKCHGNSKAELIGPEPEGADVVEQGLGWHNQNGKGVGRETMSSGIEGAWTTHPTQWDNGYFYMLFNHEWELKKSPAGAWQWEPVNIKEEDKPVDVEDPNIRHNPIMTDADMAMIKDPIYRQISERFYREPDYFAEVFAKAWFKLTHRDLGPKSRYLGPDVPQEDLIWQDPIPPVDYTLSEGEIKELEQQILASGLTVSELVCTAWDSARTFRSSDYRGGANGARIRLEPQKNWPGNEPTRLAKVLAVLENIQANFAKPVSLADLIVLGGGAAIAKAALDGGIEVNVPFLPGRGDATQAMTDAESFTPLEPIHDGYRNWLKQDYAVSPEELLLERTQLMGLTAPEMTVLIGGMRVLGTNHGGTKHGVFTDRVGVLSNDFFVNLTDMAYQWRPAGNNLYEIGDRQTGEVKWTATKVDLVFGSNSILRSYAEVYAQDDNREKFVRDFVAAWTKVMNADRFDLPRG

SEQ ID NO: 4 - protein, 753 aa, Escherichia coli
MSQHNEKNPHQHQSPLHDSSEAKPGMDSLAPEDGSHRPAAEPTPPGAQPTAPGSLKAPDTRNEKLNSLEDVRKGSENYALTTNQGVRIADDQNSLRAGSRGPTLLEDFILREKITHFDHERIPERIVHARGSAAHGYFQPYKSLSDITKADFLSDPNKITPVFVRFSTVQGGAGSADTVRDIRGFATKFYTEEGIFDLVGNNTPIFFIQDAHKFPDFVHAVKPEPHWAIPQGQSAHDTFWDYVSLQPETLHNVMWAMSDRGIPRSYRTMEGFGIHTFRLINAEGKATFVRFHWKPLAGKASLVWDEAQKLTGRDPDFHRRELWEAIEAGDFPEYELGFQLIPEEDEFKFDFDLLDPTKLIPEELVPVQRVGKMVLNRNPDNFFAENEQAAFHPGHIVPGLDFTNDPLLQGRLFSYTDTQISRLGGPNFHEIPINRPTCPYHNFQRDGMHRMGIDTNPANYEPNSINDNWPRETPPGPKRGGFESYQERVEGNKVRERSPSFGEYYSHPRLFWLSQTPFEQRHIVDGFSFELSKVVRPYIRERVVDQLAHIDLTLAQAVAKNLGIELTDDQLNITPPPDVNGLKKDPSLSLYAIPDGDVKGRVVAILLNDEVRSADLLAILKALKAKGVHAKLLYSRMGEVTADDGTVLPIAATFAGAPSLTVDAVIVPCGNIADIADNGDANYYLMEAYKHLKPIALAGDARKFKATIKIADQGEEGIVEADSADGSFMDELLTLMAAHRVWSRIPKIDKIPA

SEQ ID NO: 5 - DNA, 225 bp, Artificial Sequence
  Description of Artificial Sequence Synthetic polynucleotide
gcttgtagcaattgctactaaaaactgcgatcgctgctgaaatgagctggaattttgtccctctcagctcaaaaagtatcaatgattacttaatgtttgttctgcgcaaacttcttgcagaacatgcatgatttacaaaaagttgtagtttctgttaccaattgcgaatcgagaactgcctaatctgccgagtatgcgatcctttagcaggaggaaaaccatatg

SEQ ID NO: 6 - DNA, 49 bp, Artificial Sequence
  Description of Artificial Sequence Synthetic oligonucleotide
taacaccgtgcgtgttgactattttacctctggcggtgataatggttgc